(19) United States
(12) Patent Application Publication
(10) Pub. No.: US 2004/0147033 A1
(43) Pub. Date: Jul. 29, 2004

(54) Glycan markers for diagnosing and monitoring disease

(76) Inventors: Zachary Shriver, Boston, MA (US); Ganesh Venkataraman, Bedford, MA (US); Ram Sasisekharan, Cambridge, MA (US); Mallikarjun Sundaram, Ashland, MA (US)

Correspondence Address: FISH & RICHARDSON PC, 225 FRANKLIN ST, BOSTON, MA, 02110, US

(21) Appl. No.: 10/742,617

(22) Filed: Dec. 19, 2003

Related U.S. Application Data
(60) Provisional application No. 60/435,586, filed on Dec. 20, 2002.

(51) Int. Cl. G01N 33/48

(52) U.S. Cl. 436/87; 436/64

(57) ABSTRACT

The present invention provides ultra-sensitive methods for detecting changes in glycosylation that are correlated with pre-cancerous or early cancerous states. Because the chance of complete recovery is increased with earlier detection of cancer, the present invention provides therapeutically useful methods of early detection, diagnosis, staging and prognostication.

### CLAIM OF PRIORITY

This application claims priority under 35 USC § 119(e) to U.S. Patent Application Serial No. 60/435,586 filed on Dec. 20, 2002, the entire contents of which are hereby incorporated by reference.

### TECHNICAL FIELD

This invention relates to diagnosing and monitoring disease, and more particularly to diagnosing and monitoring cancer.

### BACKGROUND

The importance of carbohydrates in the physiology of living organisms has been recognized. Beyond their crucial role in metabolism, sugars play a role in almost every physiological process. For instance, linear sugars found on cell surfaces and attached to proteins and lipids provide characteristic cellular signatures, mediate cell-cell communications, and actively orchestrate intracellular signal transduction. Branched and linear sugars found on the surfaces of proteins and other biopolymers provide characteristic protein signatures, mediate protein localization and targeting, and actively modulate protein function and efficacy, stabilize pharmacokinetics, and can affect therapeutic (clinical) potency.

Although changes in the regulation and processing of sugars have been correlated to a number of abnormal physiologic states, a lack of sufficiently sensitive detection methods has limited the usefulness of these markers to conditions under which there are gross changes in carbohydrates, which generally correlate with extremely advanced disease states. The present invention provides novel methods having increased sensitivity, which allows for the detection of more subtle sugar changes which may be associated with earlier as well as later disease stages.

### SUMMARY

The present invention is based on the discovery of ultra-sensitive diagnostic methods for detecting changes in glycosylation that are correlated with pre-cancerous, early cancerous, or cancerous states, e.g., changes correlated with cell transformation or metastasis.

In one aspect, the present invention provides a method for evaluating a subject by providing a sample comprising a pre-selected target glycoprotein, for example, a marker for cancer, such as for example prostate specific antigen (PSA), alpha-fetoprotein (AFP), or carcinoembryonic antigen (CEA). The sample can be any bodily fluid or tissue from a subject, including but not limited to urine, blood, serum, semen, saliva, feces, or tissue, and the sample can be unconcentrated or concentrated using routine methods. The glycoprofile of the target glycoprotein is determined using a method that is sufficiently sensitive to detect a target glycoprotein in amounts less than about 1000 ng/ml e.g., less then about 500, 250, 100, 75, 50, 25, 20, 10, 5, 4, 3, 2, 1, 0.5, 0.1 ng/ml of target glycoprotein in the sample, for example, from about 0.1 ng/ml to 1 μg/ml of target glycoprotein. In some embodiments, the sample has a greater amount of the target glycoprotein than the limit of detection of the method used to determine the glycoprofile, e.g., has greater than 1000 ng/ml of the target glycoprotein. Assuming an average mass of about 20,000 Da, this is equivalent to about 5 pM-50 nM or about 5 femtomoles/ml to about 50 picomoles/mL of target glycoprotein. In some embodiments, the glycoprofile indicates that the subject has a predefined clinical status, for example, one of a set of stages, such as stages which correspond to progressive stages of a disorder, e.g., cancer, a precancerous condition, a benign condition, or no condition (“no condition” as used herein means that the subject does not have any benign, precancerous or cancerous condition associated with the preselected target glycoprotein).

In a second aspect, the present invention provides a method for evaluating a subject by providing a sample from the subject. In some embodiments, the sample can comprise any of the following: about 0.1 ng/ml to 1 μg/ml; about 5 pM-50 nM, e.g., about 5 femtomoles/ml to about 50 picomoles/ml; less than about 1 μg; or less than about 50 pmols of a pre-selected target glycoprotein. The glycoprofile of the target molecule is then determined, and in some embodiments, the glycoprofile indicates that the subject has a predefined clinical status, e.g., one of a set of stages which correspond to progressive stages of a disorder, e.g., that the subject has cancer, a precancerous condition, a benign condition, or no condition.

In a third aspect, the invention features a method for evaluating the clinical status of a subject by providing a sample from the subject, isolating a preselected target glycoprotein from the sample, e.g., by immunopurification; and contacting the target protein with an enzyme. The enzyme can be an immobilized enzyme, e.g., an enzyme bound to a bead. The enzyme can be bound to the bead using any method known in the art, such as chemically crosslinking the antibody to the bead using a bifunctional crosslinker, including but not limited to bis(sulfosuccinimidyl)suberate and/or dimethyl adipimidate. Then, the glycoprofile of the target protein is determined. In some embodiments, the glycoprofile indicates that the subject has a predefined clinical status, e.g., one of a set of stages which correspond to progressive stages of a disorder, e.g., that the subject has cancer, a precancerous condition, a benign condition, or no condition.

In one embodiment, determining the glycoprofile of a target glycoprotein can include removing one or more pre-selected glycans from said target molecule; e.g., enzymatically (using, for example, PNGase F, PNGase A, EndoH, EndoF, O-glycanase, and/or one or more proteases, e.g., trypsin, or LysC) or chemically (e.g., using anhydrous hydrazine (N) or reductive or non-reductive beta-elimination (O)).

In another embodiment, one or more experimental constraints can be applied to the glycan, such as enzyme or chemical digestion.

In some embodiments, one or more of the method steps can be repeated. This repetition can be done before, during and/or after administration of a treatment to the subject, to monitor the effectiveness of the treatment.

In some embodiments, the sample can comprise less than about 50 pmol of the target glycoprotein; less than about 10 pmol of the target glycoprotein; less than about 1.0 pmol of the target glycoprotein; less than about 0.5 pmol of the target glycoprotein; less than about 0.1 pmol of the of the target glycoprotein; less than about 0.05 pmol of the target glycoprotein; less than about 0.01 pmol of the target glycoprotein; or less than about 0.005 pmol of the target glycoprotein.

In a further embodiment, determining the glycoprofile comprises determining one or more of: the presence, concentration, percentage, composition, or sequence of one or more glycans associated with the target molecule. The glycoprofile can be determined by a method selected from CE, e.g., CE/LIF, NMR, mass spectrometry (both MALDI and ESI), and HPLC with fluorescence detection.

In some embodiments, determining the glycoprofile comprises detecting alterations in one or more of sialylation, modification of sialic acids, including sulfation, branching, presence or absence of a bisecting N-acetylglucosamine, or changes in the number of glycosylation sites. In some embodiments, determining the glycoprofile comprises detecting alterations in β1→6 branching structures, e.g., of N-linked and/or O-linked oligosaccharides. In some embodiments, determining the glycoprofile comprises detecting alterations in Lewis antigens, e.g., Lewis antigen levels, sialylation, and/or fucosylation, inter alia.

In some embodiments, the subject is suspected of having a cellular proliferative and/or differentiative disorder, such as cancer, e.g., carcinoma, sarcoma, metastatic disorders or hematopoietic neoplastic disorders, e.g., leukemias. In some embodiments, the glycoprofile indicates that the subject has cancer; has a pre-disorder condition, e.g., a precancerous condition; or has a benign condition, such as a benign tumor, benign hyperplasia, e.g., BPH; or has no condition, i.e., is normal. In some embodiments, the presence, concentration, percentage, composition, or sequence of one or more glycans indicates that the subject has cancer; has a pre-disorder condition, e.g., a precancerous condition; or has a benign condition, such as a benign tumor, benign hyperplasia, e.g., BPH. In some embodiments, the cancer is breast carcinoma, lung carcinoma, colon carcinoma, prostate cancer or hepatocellular carcinoma.

In some embodiments, the presence, concentration, percentage, composition or sequence of one or more glycans further indicates the stage of the cancer and/or the growth rate of the cancer, and/or the prognosis.

In some embodiments, the subject does not have cancer and/or has one or more benign hyperplasias, such as benign prostatic hyperplasia, or a precancerous condition e.g., a condition that is likely to progress to cancer.

In some embodiments, the subject has a PSA level of about 0-4 ng/mL, about 4-10 ng/mL or about 10-20 ng/mL or more.

In some embodiments, the subject is being screened for a disorder characterized by changes in the glycoprofile of a target protein, e.g., a cellular proliferative and/or differentiative disorder, e.g., cancer. In some embodiments, the subject has previously tested negative for the disease by another, non-sugar-based diagnostic method, e.g., physical examination, immunodiagnostic test; detection of protein levels, e.g., in blood or urine; imaging, e.g., x-ray, MRI, CAT, ultrasound; or biopsy. In some embodiments, a second, non-glycoprofile diagnostic test is also performed, e.g., before, concurrently with, or after the glycoprofile determination.

In some embodiments, the method can also include providing a reference glycoprofile, such as a reference glycoprofile correlated with known normal, benign, precancerous, or cancerous states, and comparing the glycoprofile of the target glycoprotein to the reference. The reference can be included in a database as described herein. Comparing the glycoprofile can include comparing any data determined by the methods of the present invention, including but not limited to the presence, concentration, percentage, composition or sequence of one or more selected glycans of the target glycoprotein, to the reference. This comparison allows diagnosis, staging, prognosis, or monitoring.

In a fourth aspect, the invention provides a method for monitoring a subject by providing a sample from the subject comprising a target protein; immunopurifying the target protein; contacting the target protein with immobilized enzyme; determining the glycoprofile of the target protein; and, optionally, repeating the prior steps one or more times. The repetition of steps can be done after administration of a treatment to the subject.

In a fifth aspect, the invention provides methods for determining the metastatic potential of a tumor by providing a sample from the subject; isolating a target protein by immunopurification; contacting the target protein with one or more immobilized enzyme; and determining the glycoprofile of the target protein, wherein the glycoprofile indicates the metastatic potential of the tumor.

In a sixth aspect, the invention provides a database comprising a plurality of records. Each record can include one or more of the following:

data on the glycoprofile of a target glycoprotein associated with a disorder isolated from a sample from a subject;

data on the status of the subject, e.g., whether the subject has cancer, a pre-cancerous condition, a benign condition, or no condition, and any clinical outcome data, e.g., metastasis, recurrence, remission, recovery, or death;

data on any treatment administered to the subject;

data on the subject's response to treatment, e.g., the efficacy of the treatment;

personal data on the subject, e.g., age, gender, education, etc. and/or

environmental data, such as the presence of a substance in the environment, residence in a preselected geographic area, and performing a preselected occupation. In some embodiments, the database is created by entering data resulting from determining the glycoprofile of a target glycoprotein in a sample from a subject using a method described herein.

In a seventh aspect, the invention provides a method of evaluating a subject by providing a sample from the subject; immunopurifying a target protein from the sample; and determining the glycoprofile of the target protein in the sample, wherein the glycoprofile of the target protein in the sample indicates that the subject has cancer, a precancerous condition, or a benign condition.

In an eighth aspect, the invention provides a method of evaluating a subject, such as a subject suspected of having prostate cancer, the method comprising providing a sample from said subject, immunopurifying PSA from the sample, and determining the glycoprofile of the PSA in the sample, wherein the glycoprofile of the PSA in the sample indicates that the subject has prostate cancer, metastatic cancer, prostatitis, benign prostate hyperplasia, or no condition. In some embodiments, the glycoprofile includes one or more of: a higher degree of branching as well as sialic acid; (2) different fucosylated structures; and/or (3) different chain length of antennary arms, which indicate that the subject has prostate cancer, or is at risk for developing prostate cancer. In some embodiments, the glycoprofile indicates the presence of high molecular weight glycans that are not present in a normal or reference subject, which indicates that the subject has prostate cancer, or is at risk for developing prostate cancer. In some embodiments, the glycoprofile includes the presence of a glycan of about 3300 molecular weight that is not present in a normal or reference subject, which indicates that the subject has prostate cancer, or is at risk for developing prostate cancer. The subject can have serum PSA levels of about 0-4 ng/mL; about 4-10 ng/mL; about 10-20 ng/ml; or 20 ng/mL.

In a ninth aspect, the invention provides a method of evaluating a subject, such as a subject suspected of having liver cancer, by providing a sample from said subject, immunopurifying AFP from the sample, and determining the glycoprofile of the AFP, wherein the glycoprofile of the AFP indicates that the subject has cirrhosis or HCC or no condition. The subject can have serum AFP levels of about 0-20 ng/mL; about 20-1000 ng/mL; or >1000 ng/mL.

In a tenth aspect, the invention provides a method of evaluating a subject, e.g., a subject suspected of having one or more tumors thought to arise from entodermal tissues (including cancers of the colon, stomach, lung, pancreas, liver, breast, and esophagus), by providing a sample from said subject, immunopurifying CEA from the sample, and determining the glycoprofile of the CEA, wherein the glycoprofile of the CEA indicates that the subject has or does not have has or does not have cirrhosis, inflammatory bowel disease, chronic lung disease, pancreatitis, or a cancer of the colon, stomach, lung, pancreas, liver, breast, or esophagus. The subject can have serum or plasma AFP levels of about 0-5 ng/mL; about 5-10 ng/mL; or >10 ng/mL.

In an eleventh aspect, the invention provides a method of evaluating the status of a subject by providing a sample from the subject, immunopurifying a pre-selected target protein from the sample using antibodies bound to magnetic beads, contacting the purified target protein with immobilized enzyme, and determining the glycoprofile of the target protein, wherein the glycoprofile indicates the status of the subject.

In a twelfth aspect, the invention provides a method for identifying candidate reagents capable of detecting glycoprofile differences between a first glycoprotein having a first glycoprofile and a second glycoprotein having a second glycoprofile, by contacting the first glycoprotein with one or more candidate reagents, e.g., lectins, antibodies, and/or polysaccharide-binding peptides (for instance isolated through phage display); optionally contacting the second glycoprotein with the one or more candidate reagents, e.g., lectins, antibodies, and/or polysaccharide-binding peptides; and evaluating the ability of the candidate reagents to detect glycoprofile differences between the first and second glycoproteins. In some embodiments, the glycoprofile of the first glycoprotein and/or the second glycoprotein can also be determined. In some embodiments, the first and second glycoproteins are obtained from subjects having different clinical statuses, e.g., normal, benign hyperplastic, precancerous, cancerous, metastatic, etc. In some embodiments, the first and second glycoproteins have the same protein core.

In a thirteenth aspect, the invention provides a method for identifying glycoprotein changes correlated with patient status, for example, different stages of a diseases, with different prognoses or clinical outcomes, etc., the method comprising providing samples from a plurality of subjects, e.g., subjects having the same stage of a disease and/or subjects having different stages of a disease (the stages can be determined by standard methods); determining the glycoprofile of a target glycoprotein, e.g., a preselected target glycoprotein marker for the disease; and comparing the glycoprofile of one subject with the glycoprofile of another. The glycoprofile information obtained can then be correlated to patient status. The method may also comprise repetition of the steps, e.g., to monitor the progress of a disease in an individual and/or a number of individuals. In some embodiments, the method includes monitoring the status of an individual, e.g., monitoring the rate of growth of a cancer, the efficacy of treatment, etc. The method may further include entering the information into a database as described herein.

As used herein, the term “sample” refers to any bodily fluid or tissue from a subject, including but not limited to urine, blood, serum, semen, saliva, feces, or tissue. A sample as used herein can be unconcentrated or can be concentrated using standard methods.

As used herein, the term “glycoprofile” refers to one or more properties of the glycans of a glycoprotein; for example, the glycoprofile can include, but is not limited to, one or more of the following: number or placement of glycans; number or placement of N-linked glycans; number or placement of O-linked glycans; sequence of one or more attached glycans; tertiary structure of one or more glycans, e.g., branching pattern, e.g., biantennary, triantennary, tetrantennary, and so on; number or placement of Lewis antigens; number or placement of fucosyl or sialyl groups; molecular weight or mass of the intact glycoprotein; molecular weight or mass of the glycoprotein following the application of one or more experimental constraints, e.g., digestion (enzymatic or chemical); molecular weight or mass of some or all of the glycans after being released from the glycoprotein, e.g., enzymatically or chemically; molecular weight or mass of some or all of the glycans after being released from the glycoprotein and following the application of one or more experimental constraints; mass signature; or charge. In one embodiment, the glycoprofile is determined by a method other than one which involves determining if the glycoprotein binds one or more lectins or antibodies.

As used herein, “target protein” or “target glycoprotein” refers to a glycoprotein which demonstrates one or more changes in glycoprofile that can be correlated with the onset, state, progression, or prognosis of a disorder, e.g., a proliferative and/or differentiative disorder. The amino acid, e.g., non-sugar, part of the glycoprotein is referred to as the “core protein.” The target glycoprotein can be preselected, for example, on the basis of a risk factor, e.g., environmental or genetic risk factor, for a particular disorder, or on the basis of a previous test, e.g., a non-sugar based test, a blood test, biopsy, physical examination, etc., indicating the possibility that the subject has a particular disorder. Then the glycoprotein target associated with that disorder can be selected and the glycoprofile determined as described herein.

Examples of proliferative and/or differentiative disorders include cancer, e.g., carcinomas, sarcomas, metastatic disorders or hematopoietic neoplastic disorders, e.g., leukemias, as well as proliferative skin disorders, e.g., psoriasis or hyperkeratosis. Other myeloproliferative disorders include polycythemia vera, myelofibrosis, chronic myelogenous (myelocytic) leukemia, and primary thrombocythaemia, as well as acute leukemia, especially erythroleukemia, and paroxysmal nocturnal haemoglobinuria. Metastatic tumors can arise from a multitude of primary tumor types, including but not limited to those of prostate, colon, lung, breast and liver origin.

As used herein, the terms “cancer,” “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. “Benign hyperproliferative” cells can include non-malignant tumor cells, such as are associated with benign prostatic hyperplasias, hepatocellular adenomas, hemangiomas, focal nodular hyperplasias, angiomas, dysplastic nevi, lipomas, pyogenic granulomas, seborrheic keratoses, dermatofibromas, keratoacanthomas, keloids, and the like.

The terms “cancer” or “neoplasms” include malignancies of the various organ systems, such as affecting lung, breast, thyroid, lymphoid, gastrointestinal, and genitourinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L., Ball, E. D., Foon, K. A. (1991) Immune markers in hematologic malignancies. Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemiallymphoma (ATL), cutaneous T-cell lymphoma (CTCL), large granular lymphocytic leukemia (LGF), Hodgkin's disease and Reed-Sternberg disease.

As used herein the term “pre-cancerous” refers to a condition that is likely to develop into cancer if left untreated. Pre-cancerous conditions in general may be associated with, for example, a typical hyperplasia, a typical proliferation, dysplasia, carcinoma in situ, or intraepithelial neoplasia, inter alia, but are generally not associated with metastatic disease.

As used herein “early cancer” refers to a condition that is cancerous but has not significantly progressed, e.g., is in an early stage. In general, early stage cancer has not significantly metastasized, or has not metastasized at all.

The present invention has a number of advantages. For instance, the methods described herein allow the identification of changes in glycosylation that are associated with transformation and/or metastasis. The present methods allow this identification to be made at a much earlier stage than previously possible. Further, the present invention provides methods for diagnosing patients at a much earlier stage, thus enhancing the efficacy of, and aiding in the selection and monitoring of, treatments. The present methods also provide for the screening of individuals who are not even suspected of having cancer, including individuals who are at risk for cancer due to, for example, genetic or environmental factors.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a drawing of the glycan structure present on normal prostate serum antigen (PSA).

FIG. 2 is a drawing of the basic branching patterns of N-linked sugars.

FIG. 3 is an illustration of mass-identity relationships for the branching patterns of PSA.

FIG. 4 is a photograph of a gel showing the results of PAGE analysis of oligosaccharides derived from normal and transformed PSA (from LNCaP cells). ANTS labeled samples were separated by gel electrophoresis. Lane 1, dextran standard (Glyko); lane 2, asialobiantennary oligosaccharide without fucose; lane 3, asialobiantennary oligosaccharide with fucose; lane 4, asialotriantennary oligosaccharide marker (2,2,6); lane 5, oligosaccharides from normal PSA treated with sialidase; lane 6, oligosaccharide released from transformed PSA.

FIG. 5 is a mass spectrogram of whole PSA from normal human serum.

FIG. 6A is a mass spectrogram of intact glycans purified from PSA.

FIG. 6B is a mass spectrogram of sialidase-treated glycans purified from PSA

FIG. 6C is a mass spectrogram of galactosidase-treated glycans purified from PSA

FIG. 6D is a mass spectrogram of hexosaminidase-treated glycans purified from PSA

FIG. 7 is an illustration of the structure of the glycans of PSA, as determined from the mass spectrometry profiles as seen in FIGS. 6A-6D.

FIG. 8A is a flowchart illustrating a method for purifying PSA from blood.

FIG. 8B is a mass spectrogram of glycans isolated from PSA from cancer patients.

### DETAILED DESCRIPTION

The present invention provides ultra-sensitive methods for detecting changes in glycosylation that are correlated with pre-cancerous, early cancerous, or cancerous states, e.g., changes that accompany cell transformation or metastasis. Because the chance of complete recovery is increased with earlier detection of cancer, the present invention provides therapeutically useful methods of early detection, diagnosis, staging and prognostication.

Aberrant glycosylation occurs in essentially all types of experimental and human cancer. Among others, changes in β1→6 GlcNAc branching structure and order of N-linked glycans, changes in sialation of O-linked TN-antigen and Thomsen-Friedenriech or T-antigen structures, and changes in expression levels of sialated and unsialated Lewis factors (sialyl-Lex, sialyl-Lea, and Ley) have all been correlated to tumor progression.

In general, the carbohydrate moiety of any N-linked glycoprotein can be placed in one of three major categories on the basis of the structure and location of the monosaccharide added to this trimannosyl core: high mannose, hybrid or complex. For all of these structures, the link to the protein is through the amino acid asparagine (N-linked). In N-linked sugars the reducing terminal core is strictly conserved (Man3GlcNAc2) and the glycosylamine linkage is always via a GlcNAc residue. The large diversity of N-linked oligosaccharides arises from variations in the oligosaccharide chain beyond the core motif. First, there can be differential extension of the biantennary arms of the core. Second, variation can arise from increased branching resulting in tri- and tetrantennary structures. In this case, several N-acetylglucosaminyl transferases can act on the biantennary structure to form more highly branched oligosaccharides. Finally, other residues can be added to the nascent glycan chain including α1→6 ficosylation of the core N-acetylglucosamine residue, and a α1→3 fucosylation of antennary N-acetylglucosamine residues.

O-linked glycans attach to proteins by an O-glycosidic bond to serine or threonine on the peptide chain. Unlike N-linked sugars, O-linked sugars are based on a number of different cores, giving rise to great structural diversity. O-linked glycans are generally smaller than N-linked, and there is no consensus motif for locating O-linked glycosylation on the protein.

Changes in glycosylation patterns are known to alter the specificity and/or structure of proteins and as a consequence their function, and changes in glycosylation have been long thought to be markers of tumor progression. Changes in mucin structure have been exploited as general tumor markers for diagnosis, immunotherapy and development of potential cancer vaccines (Syrigos et al., Anticancer Res 19:5239-44 (1999); Graham et al., Cancer Immunol Immunother 42:71-80 (1996)). Several experiments have pointed to an increased number of β1→6 branchings of N-linked sugars in tumor cells and in metastases of murine melanomas and fibrosarcomas (Kawano et al., Glycobiology 1:375-385(1991); Bruyneel et al., J. Cell. Sci. 95:279-86 (1990)). Furthermore, the biological regulation of branched sugar formation appears to be altered in several cancerous cells resulting in a shift towards higher branched sugars (Takano et al., Glycobiology 4:665-74 (1994); Dennis et al., Semin. Cancer Biol. 2:411-20 (1991)). Many cancer types produce or overexpress enzymes, such as N-acetylglucosaminyl transferases IV and V, to form tri- and tetrantennary “aberrant” structures (Mori et al., J Gastroenterol. Hepatol. 13:610-9 (1998); Naitoh et al., J. Gastroenterol. Hepatol. 14:436-45 (1999); Guo et al., J. Cell. Biochem. 79:370-85 (2000)). It should be noted that the glycosylation differences can either be dramatic (as in changes in the number of branches on the sugar chain i.e. bi-antennary to tri and tetra-antennary chains) or subtle variations in terminal or internal residues.

Among the glycoproteins that have been investigated for use as diagnostic markers of cancer are α-fetoprotein (AFP) for hepatocellular carcinoma (HCC), mucin-1 (MUC1) for breast cancer, prostate specific antigen (PSA) for prostate cancer, and carcinoembryonic antigen (CEA) for tumors thought to arise from entodermal tissues, including cancers of the colon, stomach, lung, pancreas, liver, breast, and esophagus. However, to date, these methods of diagnosis have been limited by the technology available for evaluating the markers. For instance, although generally elevated PSA levels (above about 4 ng/ml) can be indicative of prostate cancer, increased PSA (about 4-10 ng/ml) can be the result of non-malignant conditions including prostatitis and benign prostate hyperplasia, or BPH. The fact that both benign and malignant prostatic growth leads to increases in plasma levels of PSA confounds its use as an indicator of cancer initiation, progression, and stage. Thus, while the PSA test has revolutionized the detection of prostate cancer and has provided a tool to estimate the efficacy of cancer treatments, it leads to a large number of false positives and is most likely the single most important factor in the unnecessary treatment of many in the population.

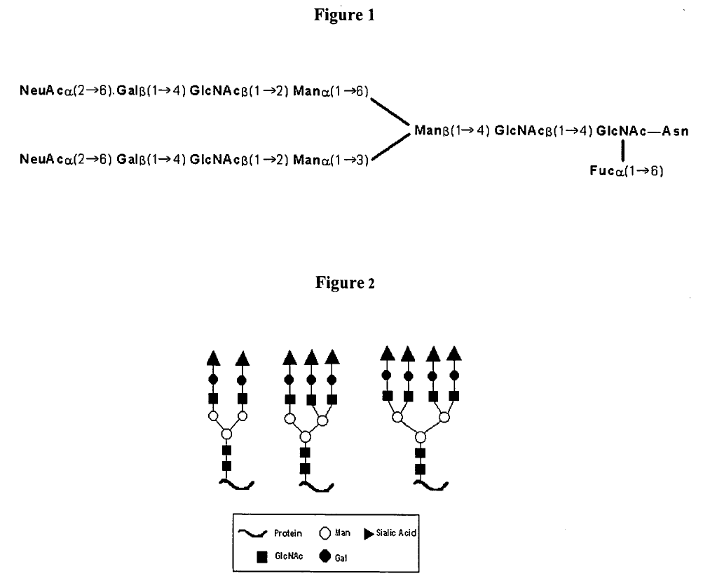

Like many proteins, PSA is a glycoprotein, with a molecular weight range from about 26,000 to 34,000 Da depending on the technique used to characterize the protein as well as the procedure used to isolate it. PSA typically contains one N-linked carbohydrate chain attached to asparagine 45 of the polypeptide chain. A majority of PSA isolated from normal human seminal fluid appears to contain a complex bi-antennary carbohydrate chain (carbohydrate chain with one branched structure) that is terminally capped by sialic acid and contains a fucose linked 1→6 to a core N-acetylglucosamine, as shown in FIG. 1 (Belanger et al., Prostate 27:187-97 (1995)). As such, human PSA is composed of 7 to 12% (by mass) carbohydrate on average. However, it has been observed that several isoforms of PSA exist in serum that differ only in the structure of the carbohydrate chain attached to asparagines (Guo et al., J. Cell. Biochem. 79:370-85 (2000)). The differences in the structure of the carbohydrate may be correlated to changes in disease status from benign to malignant (Prakash and Robbins, Glycobiology 10(2):174-176 (2000)).

α-fetoprotein (AFP) is a normal fetal serum glycoprotein synthesized by the liver, yolk sac, and gastrointestinal tract of the developing fetus with sequence homology to albumin. Although it is a major component of fetal plasma, AFP clears rapidly from the circulation after birth, and in healthy adults less than 10 μg/L is found in the circulation. AFP is elevated in normal pregnancy and in benign liver disease such as hepatitis and cirrhosis, as well as in cancer, particularly hepatocellular and germ cell (nonseminoma) carcinoma and testicular germ cell tumors, and less commonly in other malignancies such as pancreatic cancers, gastric cancers, colonic cancers, and bronchogenic cancers; like PSA, AFP levels can be used to grossly distinguish between benign and malignant conditions; elevations up to about 500 ng/ml are generally not associated with malignancies. AFP is in use as a diagnostic and therapeutic tool for use in HCC. Differences in sialation and fucosylation of AFP have been detected that correlate with the presence of malignancy (Naitoh et al., J. Gastroent. Hep. 14:436-445 (1999)).

Carcinoembryonic antigen (CEA) is a complex immunoglobulin-like glycoprotein of about 20 kD that is associated with the plasma membrane of tumor cells, from which it may be released into the blood. Although it was first identified in colon cancer, elevated CEA blood levels are not specific for colon cancer or for malignancy in general; elevated CEA levels are detected in a variety of cancers other than colonic, including pancreatic, gastric, lung, and breast, as well as benign conditions including cirrhosis, inflammatory bowel disease, chronic lung disease, and pancreatitis. Confounding the issue, CEA was found to be elevated in up to 19 percent of smokers and in 3 percent of a healthy control population, making simple CEA levels not useful for diagnostic purposes. Importantly, differences have been observed not only in the carbohydrate composition of CEA in normal versus cancerous colon tissues (Garcia et al., Cancer Res. 51(20):5679-86 (1991)), but also in CEA from different tumor sources, both in total % carbohydrate, and mole % of the individual sugars (DeYoung et al., Aust J Exp Biol Med Sci. 56(3):321-31 (1978)).

A number of other proteins have been described which have altered glycosylation patterns that make them potentially useful markers for malignancy, including α-1-antitypsin and transferrin, which demonstrate altered fucosylation in HCC (Naitoh et al., supra). Other potential markers include insulin-like growth factor-1 (IGF-1); human chromic gonadotropin (HCG), particularly the beta subunit; CA125, a marker for some breast cancers; guanylyl cyclase-C (GC-C), a marker for some colorectal, bladder, and stomach cancers; Nuclear matrix proteins NMP 22 and 48, NMP22 for bladder cancers and NMP48 for prosate cancers; alpha-methylacyl-CoA racemase (AMACR), a marker for some prostate cancers; and CA19-9 (pancreatic and gastrointestinal, e.g., stomach cancers), CA242 (pancreatic and lung cancers), CA72-4 (colorectal and ovarian cancers) and CA50 (pancreatic and bladder cancers)(see Carpelan-Holmstrom et al., Anticancer Res. 22(4):2311-6 (2002); Chang et al., J. Natl. Cancer Inst. 94(22):1697-703 (2002); Sedlaczek et al., Cancer 95(9):1886-93 (2002); Bubley et al., J. Urol. 168(5):2249-52 (2002); Louhimo et al., Int. J. Cancer 101(6):545-8 (2002); Rodriguez et al., Cancer 95(3):670-1 (2002); Lahme et al., Urol. Int. 66(2):72-7 (2001)).

Until now, all of these potentially useful markers have been limited to use in cases of extremely advanced cancers or in non-physiologic in vitro systems due to the lack of sensitivity of the detection methods of the prior art. Chromatographic and electrophoretic techniques, in combination with enzymatic or chemical cleavage, have been developed to identify and quantify the monomeric saccharide composition of oligosaccharide chains (Chen et al., Glycobiology 8:1045-52 (1998); Raju et al., Glycobiology 10:477-86 (2000)). Fluorophore Assisted Carbohydrate Analysis (FACE), as the name suggests, involves labeling the oligosaccharide with a fluorescent probe and subsequent separation of glycan structures on a polyacrylamide gel electrophoresis (Frado et al., Electrophoresis 21:2296-308 (2000); Yang et al., Biotechnol Prog 16:751-9 (2000)). While the FACE and HPLC techniques are very powerful, a serious limitation is the need for microgram amounts of material for characterization. Furthermore, the labeling protocols to detect oligosaccharide structures and the gel/HPLC separation techniques are lab intensive. Thus, there is a clear need for a method that is applicable to small quantities of sample material. The present invention, requiring only pico- to femtomoles of material, provides such a method.

In some embodiments, the methods of the present invention can include determining the glycoprofile of a glycoprotein. The properties can be determined by analyzing the glycans of the intact glycoprotein, by releasing the glycans from the glycoprotein before analysis, or by digesting the intact glycoprotein and analyzing the glycans attached to one or more of the resulting glycopeptide fragments. Properties of the glycans which can be determined include: the mass of part or all of the saccharide structure, the charges of the chemical units of the saccharide, identities of the chemical units of the saccharide, confirmations of the chemical units of the saccharide, total charge of the saccharide, total number of sulfates of the saccharide, total number of acetates, total number of phosphates, presence and number of carboxylates, presence and number of aldehydes or ketones, dye-binding of the saccharide, compositional ratios of substituents of the saccharide, compositional ratios of anionic to neutral sugars, presence of uronic acid, enzymatic sensitivity, linkages between chemical units of the saccharide, charge, branch points, number of branches, number of chemical units in each branch, core structure of a branched or unbranched saccharide, the hydrophobicity and/or charge/charge density of each branch, absence or presence of GlcNAc and/or fucose in the core of a branched saccharide, number of mannose in an extended core of a branched saccharide, presence or absence or sialic acid on a branched chain of a saccharide, the presence or absence of galactose on a branched chain of a saccharide.

A property of a glycan can be identified by any means known in the art. The procedure used to identify a property may depend on the type of property; methods include, but are not limited to, capillary electrophoresis (CE), NMR, mass spectrometry (both MALDI and ESI), and HPLC with fluorescence detection. For example, molecular weight can be determined by several methods including mass spectrometry. The use of mass spectrometry for determining the molecular weight of glycans is well known in the art. Mass spectrometry has been used as a powerful tool to characterize polymers such as glycans because of its accuracy (±1 Dalton) in reporting the masses of fragments generated (e.g., by enzymatic cleavage), and also because only pM sample concentrations are required. For example, matrix-assisted laser desorption ionization mass spectrometry (MALDI-MS) has been described for identifying the molecular weight of polysaccharide fragments in publications such as Rhomberg, et al., PNAS USA 95, 4176-4181 (1998); Rhomberg, et al., PNAS USA 95, 12232-12237 (1998); and Ernst, et al. PNAS USA 95, 4182-4187 (1998). Other types of mass spectrometry known the art, such as electron spray-MS, fast atom bombardment mass spectrometry (FAB-MS) and collision-activated dissociation mass spectrometry (CAD) can also be used to identify the molecular weight of the glycan or glycan fragments. The compositional ratios of substituents or chemical units (quantity and type of total substituents or chemical units) can be determined using methodology known in the art, such as capillary electrophoresis. A glycan can be subjected to an experimental constraint such as enzymatic or chemical degradation to separate each of the chemical units of the glycans, or fragments of the glcyans. These units then can be separated using capillary electrophoresis to determine the quantity and type of substituents or chemical units present in the glycan.

Mass spectrometry data is a valuable tool to ascertain information about the glycan fragment sizes after the glycan has undergone degradation with enzymes or chemicals. After a molecular weight of a glycan is identified, it can be compared to molecular weights of other known glycans. Because masses obtained from the mass spectrometry data are accurate to one Dalton (1D), the size of one or more glycan fragments obtained by enzymatic digestion can be precisely determined, and a number of substituents (i.e., sulfates and acetate groups present) can be determined. One technique for comparing molecular weights is to generate a mass line and compare the molecular weight of the unknown glycan to the mass line to determine a subpopulation of glycans which have the same molecular weight. A “mass line” as used herein is an information database, preferably in the form of a graph or chart which stores information for each possible type of glycan having a unique sequence based on the molecular weight of the glycan. Thus, a mass line can describe a number of glycans having a particular molecular weight. For example, a two-unit polysaccharide (i.e., disaccharide) has 32 possible polymers at a molecular weight corresponding to two saccharides. Thus, a mass line can be generated by uniquely assigning a particular mass to a particular length of a given fragment (all possible di, tetra, hexa, octa, up to a hexadecasaccharide), and tabulating the results.

In addition to molecular weight, other properties can be determined using methods known in the art. The compositional ratios of substituents or chemical units (quantity and type of total substituents or chemical units) can be determined using methodology known in the art, such as capillary electrophoresis. A glycan can be subjected to an experimental constraint such as enzymatic or chemical degradation to separate each of the chemical units of the glycans. These units then can be separated using capillary electrophoresis to determine the quantity and type of substituents or chemical units present in the glycan. Additionally, a number of substituents or chemical units can be determined using calculations based on the molecular weight of the glycan. A number of experimental constraints can be applied to aid in the determination of the glycoprofile; for instance, the sugar can be degraded or modified by enzymatically removing one or more chemical unit(s) of the polysaccharide, e.g., one or more of a sialic acid, fucose, galactose, glucose, xylose, GlcNAc, and/or a GalNAc can be removed from the polysaccharide moiety. Examples of enzymes which can be used to remove a chemical unit from the polysaccharide moiety include: α-galactosidase to cleave a α1→3 glycosidic linkage after a galactose, β-galactosidase to cleave a β1→4 linkage after a galactose, an α2→3 sialidase to cleave a α2→3 glycosidic linkage after a sialic acid, an α2→6 sialidase to cleave after an α2→6 linkage after a sialic acid, an α1→2 fucosidase to cleave a α1→2 glycosidic linkage after a fucose, a α1→3 fucosidase to cleave a α1→3 glycosidic linkage after a fucose, an α1→4 fucosidase to cleave a α1→4 glycosidic linkage after a fucose, an α1→6 fucosidase to cleave an α1→6 glycosidic linkage after a fucose, a N-acetylglucosaminidase to cleave a β1→2, a β1→4 or β1→6 linkage after a GlcNAc.

The structure and composition of the saccharide moiety can be analyzed, for example, by enzymatic degradation. For each type of monosaccharide and the various types of linkages between a particular monosaccharide and a polysaccharide chain, there exists a modifying enzyme. For example, galactosidases can be used to cleave glycosidic linkages after a galactose. Galactose can be present in a polysaccharide chain through an α1→3 glycosidic linkage or a β1→4 linkage. α-Galactosidase can be used to cleave α1→3 glycosidic linkages after a galactose and β-galactosidase can be used to cleave a β1→4 linkage after a galactose. Sources of β-galactosidase include S. pneumoniae. In addition, various sialidases can be used to specifically cleave an α2→3, an α2→6, an α2→8, or an α2→9 linkage after a sialic acid. For example, sialidase from A. urefaciens cleaves all sialic acids whereas other enzymes show a preference for linkage position. Sialidase (S. pneumoniae) cleaves α2→3 linkages almost exclusively whereas Sialidase II (C. perringens) cleaves α2→3 and α2→6 linkages only. Fucose can be linked to a polysaccharide by any of an α1→2, α1→3, α1→4, and α1→6 glycosidic linkage, and fucosidases which cleave each of these linkages after a fucose can be used. α-Fucosidase II (X. manihotis) cleaves only α1→2 linkages after fucose whereas α-fucosidase from bovine kidney cleaves only α1→6 linkages. GlcNAc can form three different types of linkages with a polysaccharide chain. These are a β1→2, a β1→4 and a β1→6 linkages. Various N-acetylglucosaminidase can be used to cleave GlcNAc residues in a polysaccharide chain. β-N-Acetylhexosaminidase from Jack Bean can be used to cleave non-reducing terminal β2,3,4,6 linked N-acetylglucosamine, and N-acetylgalactosamine from oligosaccharides whereas alpha-N-Acetylgalactosaminidase (Chicken liver) cleaves terminal alpha 1→3 linked N-acetylgalactosamine from glycoproteins. Other enzymes such as aspartyl-N-acetylglucosaminidase can be used to cleave at a beta linkage after a GlcNAc in the core sequence of N-linked oligosaccharides.

Enzymes for degrading a polysaccharide at other specific monosaccharides such as mannose, glucose, xylose and N-acetylgalactosamine (GalNAc) are also known.

Degrading enzymes are also available which can be used to determine branching identity, i.e., is a polysaccharide mono-, bi-, tri- or tetrantennary. Various endoglycans are available which cleave polysaccharides having a certain number of branches but do not cleave polysaccharides having a different number of branches. For example, EndoF2 is an endoglycan that clips only biantennary structures. Thus, it can be used to distinguish biantennary structures from tri- and tetrantennary structures.

In addition, modifying enzymes can be used to determine the presence and number of substituents of a chemical unit. For example, enzymes can be used to determine the absence or presence of sulfates using, e.g., a sulfatase to remove a sulfate group or a sulfotransferase to add a sulfate group.

Glucuronidase and iduronidase can also be used to cleave at the glycosidic linkages after a glucuronic acid and an iduronic acid, respectively. In a similar manner, enzymes exist that cleave galactose residues in a linkage specific manner and enzymes that cleave mannose residues in a linkage specific manner.

The property of the glycan that is detected by this method can also be any structural property of a glycan or unit. For instance, the property of the glycan can be the molecular mass or length of the glycan. In other embodiments the property can be the compositional ratios of substituents or units, type of basic building block of a polysaccharide, hydrophobicity, enzymatic sensitivity, hydrophilicity, secondary structure and conformation (i.e., position of helices), spatial distribution of substituents, linkages between chemical units, number of branch points, core structure of a branched polysaccharide, ratio of one set of modifications to another set of modifications (i.e., relative amounts of sulfation, acetylation or phosphorylation at the position for each), and binding sites for proteins.

Methods of identifying other types of properties are easily identifiable to those of skill in the art and generally can depend on the type of property and the type of glycan; such methods include, but are not limited to capillary electrophoresis (CE), NMR, mass spectrometry (both MALDI and ESI), and HPLC with fluorescence detection. For example, hydrophobicity can be determined using reverse-phase high-pressure liquid chromatography (RP-HPLC). Enzymatic sensitivity can be identified by exposing the glycan to an enzyme and determining a number of fragments present after such exposure. The chirality can be determined using circular dichroism. Protein binding sites can be determined by mass spectrometry, isothermal calorimetry and NMR. Linkages can be determined using NMR and/or capillary electrophoresis. Enzymatic modification (not degradation) can be determined in a similar manner as enzymatic degradation, i.e., by exposing a substrate to the enzyme and using MALDI-MS to determine if the substrate is modified. For example, a sulfotransferase can transfer a sulfate group to an oligosaccharide chain having a concomitant increase of 80 Da. Conformation can be determined by modeling and nuclear magnetic resonance (NMR). The relative amounts of sulfation can be determined by compositional analysis or approximately determined by raman spectroscopy.

Methods for identifying the charge and other properties of polysaccharides have been described in Venkataraman, G., et al., Science, 286, 537-542 (1999), and U.S. patent application Ser. Nos. 09/557,997 and 09/558,137, both filed on Apr. 24, 2000, which are hereby incorporated by reference. Other suitable methods for use as described here are known to those skilled in the art. See, for example, Keiser, et al., Nature Medicine 7(1), 1-6 (January 2001); Venkataraman, et al., Science 286, 537-542 (1999). See also, U.S. Pat. No. 6,190,522 to Haro, 5,340,453 to Jackson, and 6,048,707 to Klock, for specific techniques that can be utilized.

In the method of capillary gel-electrophoresis, reaction samples can be analyzed by small-diameter, gel-filled capillaries. The small diameter of the capillaries (50 microns) allows for efficient dissipation of heat generated during electrophoresis. Thus, high field strengths can be used without excessive Joule heating (400 V/m), lowering the separation time to about 20 minutes per reaction run, therefore increasing resolution over conventional gel electrophoresis. Additionally, many capillaries can be analyzed in parallel, allowing amplification of generated glycan information. In particular, capillary electrophoresis coupled with Laser Induced Fluorescence detection (CE-LIF) can be used to achieve accurate structural determinations. (Krylov et al., J. Chromatogr. B741:31-35 (2000); Song et al., Anal. Biochem. 304(1):126-9 (2002); Monsarrat et al., Glycobiology 9(4):335-42 (1999)).

In one aspect, the present method can include the construction and use of a database comprising a plurality of records containing data regarding known glycan molecules having known properties, when analyzed using one or more techniques for analysis, e.g., as described in U.S. patent application Ser. No. 10/244,805. For example, the known glycans can be target glycoproteins, saccharides, oligosaccharides or polysaccharides of known composition, structure and molecular weight. The properties can be the data obtained using a technique such as capillary electrophoresis, high pressure liquid chromatography (HPLC), gel permeation and/or ion exchange chromatography, nuclear magnetic resonance (NMR), modification with an enzyme such as digestion with an exoenzyme or endoenzyme, chemical digestion, or chemical modification, inter alia. The process can be performed for the entire molecule or a portion thereof. The results can also be further quantitated. Each record in the database can include one or more of the following: data on the status of the subjects from whom the known glycans were isolated, e.g., normal, cancerous, pre-cancerous, benign; data on the correlation of one or more properties of the glycan to the subjects' status; prognostic data; therapeutic data (such as the administration of a given compound and the subsequent effect of the compound); data on the growth rate of any cancers, etc. In some embodiments, the record can include data on one or more of: the presence of a treatment (e.g., the administration of a compound e.g., a drug (e.g., a hormone), vitamin, food or dietary supplement); the presence of an environmental factor (e.g., the presence of a substance in the environment); the presence of a genetic factor or physical factor such as age.

The database can be any kind of storage system capable of storing the various data for each of the records as described herein. For example, the database can be a flat file, a relational database, a table in a database, an object in a computer readable volatile or non-volatile memory, data accessible by computer program, such as data stored in a resource fork of an application program file on a computer readable storage medium. Preferably, the database is in a computer readable medium (e.g., a computer memory or storage device).

Once the ultrasensitive methods of the present invention have been used to determine the nature of the changes in glycosylation that accompany the transformation process, the information derived can be used to develop other diagnostic tools, such as kits based on ELISA and/or lectin-binding techniques. Thus the information derived using the methods described herein could be used to provide the information for the development of other accurate assays of glycosylation changes with the onset of cancer. In addition, the methods of the present invention can be used to correlate the mass and identity of the glycans on a target protein with a given disease state or stage, thus allowing for rapid staging using only a simple mass determination. This information is useful to physicians, for example in selecting treatments, e.g., directing a physician to choose a particular treatment course, and/or allowing the physician to monitor the progress of a selected treatment course. For example, if the glycoprofile of the target glycoprotein indicates that a cancer is unlikely to become metastatic, the physician can choose not to use chemotherapy or radiation therapy.

### EXAMPLES

The invention is further described in the following examples, which do not limit the scope of the invention described in the claims.

Materials and Methods.

Characterization of Immunopurified Target Protein:

Target protein and glycan purity was examined by Western blotting followed by silver staining (to detect protein) and/or by glycoprotein ECL chemiluminescence (to detect carbohydrates) (Amersham). In the latter assay, carbohydrate residues are oxidized with periodate and then linked to a biotin hydrazide. The signal was developed as in other chemiluminescence detection systems according to the manufacturer's directions. Proteins that are not glycosylated give no signal. These detection systems are suited to examination of the eluates from immobilized antibody columns, and will provide information needed for further characterization. Once a clean protein band was detected in the material isolated, we proceeded directly to MS sequencing. Immunopurification is typically sufficient for glycotyping.

Carbohydrate Structure Determination by MALDI-MS of Intact Proteins or Peptide Fragments:

Once the protein recovered in the step above was determined to be relatively pure, the intact protein was then examined by MALDI-MS directly. In addition, peptides derived from using suitable proteolytic enzymes can be analyzed; a small peptide containing a carbohydrate moiety which is produced by a suitable proteolytic enzyme (e.g. clostripain or chymotrypsin) can be isolated and examined by MS. These glycopeptides could be about 9-13 amino acids long and thus have a molecular weight in the range of about 1000-4500 Da, a region where mass spectrometric data can be obtained more easily, accurately and with high sensitivity (requiring less than a picomole of material). As stated earlier, MALDI-MS is very sensitive and requires only a few picomoles or less of material. The mass accuracy was in the order of about 0.1-0.01%. In the case of glycopeptides, the analysis is typically completed in the positive mode using either 2,5-dihydroxybenzoic acid or (α-cyano-4-hydroxycinnamic acid). Then, accelerating voltage and grid voltage of the machine are systematically changed to maximize the signal-to-noise ratio.

Preparation of Oligosaccharides for MS Analysis or Sequencing:

N-linked glycans were released from affinity purified proteins by incubation with PNGase F (New England Biolabs). Using PNGase F covalently bonded to amine-derivatized magnetic beads (Pierce), approximately 1-10 μg or more of glycoprotein was digested to yield 50 ng-1 μg of polysaccharides. (Smaller or larger amounts can also be used, and other enzymes can also be bound to beads, e.g., by chemically crosslinking to the bead using a bifunctional crosslinker, such as bis(sulfosuccinimidyl)suberate or dimethyl adipimidate). The protein was first denatured for 10 minutes at 95° C., then incubated with PNGase F overnight at 37° C. A 3× volume of cold ethanol was then added to the sample and incubated on ice for 1 hour to precipitate the protein, leaving the released glycans in solution. After centrifuging for 5 minutes, the supernatant was collected and dried on a SpeedVac. Dried glycans were then resuspended in water and purified on a GlycoClean H activated carbon cartridge (Glyko). The eluted sample was then lyophilized to dryness, and resuspended in 100 μl of water for sequencing or MALDI analysis, to give a final concentration of approximately 10 μM/L.

Carbohydrate Structure Determination by MALDI-MS of Isolated Glycans:

N-linked glycans were analyzed using a 2,5-dihydroxybenzoic acid matrix with 300 mM spermine in water. One microliter (1 μl) of a glycan sample, of approximately 50 femtomoles-100 pmoles, generally in the range of 5-20 pmoles, was applied to the MALDI-MS plate, immediately followed by 1 μl of saturated matrix solution. The sample was then allowed to dry prior to analysis (Mechref and Novotny, Journal of the American Society for Mass Spectrometry 9:1293-1302 (1998); Mechref and Novotny, Analytical Chemistry 70:455-463 (1998)). Alternatively, saccharide complexation with a peptide can be used (Venkataraman et al., Science 286:537-42. (1999); Rhomberg et al, Proc. Natl. Acad. Sci. USA 95:4176-81 (1998)). For sequence analysis, the appropriate glycosidase was added (for example, sialidase, β-galactosidase or N-acetylhexosamidase) in sodium acetate buffer according to manufacturer's instructions (Glyko, Inc.) and the mass of the saccharide structures was measured after appropriate incubation procedures. Sequencing of N-linked oligosaccharides from serum-derived PSA with MALDI-MS involves the following strategy: an array of glycosidases can be used to read the sequence from the terminal non-reducing end to the N-acetylglucosamine N-linked to the asparagine residue.

Determination of Mass-Identity Relationships:

Once the mass of the glycans on the target proteins in the samples has been determined, this mass is then associated with the identity of those glycans using methods known in the art, see for example U.S. Pat. No. 5,607,859, U.S. Ser. No. 09/558,137 WO 00/65521. As one example, the mass-identity relationship for normal PSA would be determined as follows.

Shown in Table 1 are the molecular weights of the different building blocks of an oligosaccharide chain typically found on N-linked glycosylation sites. As one example, PSA derived from normal tissue has these building blocks arranged in a specific sequence, i.e., as shown in FIG. 1. If the biochemical pathways of branched sugar formation are different in the tumor cells, then additional branches can be added to the PSA oligosaccharide core. The introduction of an additional branch (i.e., formation of a triantennary structure in correlation with the onset of malignancy) will generally result in a mass change, e.g., a mass change of approximately 657 Da above that of the PSA oligosaccharide derived from normal PSA. Similarly, the mass of a tetrantennary saccharide will generally increase by 1,022 Da compared to the normal biantennary saccharide structure present on PSA. The mass differences of the oligosaccharides can be easily monitored using a MALDI-MS technique as described herein.

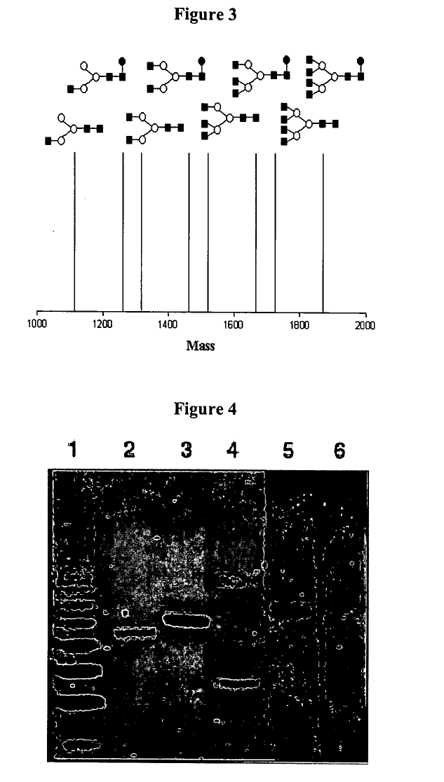

As one example, PSA isolated from serum (normal) generally has a predominant glycosylation of the biantennary type with a mass of 2370.2 Da. However, PSA isolated form cancer cells (e.g., LnCaP cells) generally has the 2370.2 Da biantennary structure, plus additional species corresponding to triantennary (3026.8 Da) and tetrantennary (3392.1 Da) saccharides. This characteristic difference in the mass spectrum of PSA from normal and cancer cells can be used to establish a “mass-identity” correlate, as shown in FIG. 3. This can be done for any target protein. It is important to note that while each of the peaks in this mass-identity spectrum represents a class of molecules (bi-, tri- or tetra-antennary), subtle variations within each of these groups can result in the further splitting of these peaks. For example, the masses mentioned above were calculated including the presence of terminal sialic acid residues for each of the chains. This may or may not always be the case. For instance, only two (instead of three) of the chains in a triantennary structure might have terminal sialic acids. In this case, the mass will correspondingly change, and such changes are readily detected using the MS methods described herein. A mass signature of the oligosaccharide representing the ‘normal’ target glycoprotein, e.g., PSA, as compared to target glycoprotein, e.g., PSA, from tumor cells can be easily obtained from this analysis. Reproducible differences corresponding to systematic changes in glycan metabolism within cancer cells, e.g., prostate cancer cells, e.g., LNCaP cells, will be identifiable using the present methods.

TABLE 1
Table of common monomers found in N linked
glycoproteins and their molecular weights.

| IDENTITY OF MONOMER | MASS |
|---|---|
| Glucose | 180.2 |
| Galactose | 180.2 |
| Mannose | 180.2 |
| Fucose | 164.2 |
| N-Acetyl-Glucosamine | 221.2 |
| N-Acetyl Galactosamine | 221.2 |
| Xylose | 150.1 |
| N-Acetyl Neuraminic Acid | 309.3 |

Correlation of Mass-Identity Relationships with Disease State or Stage:

Samples from subjects with different known disease states and stages are analyzed, e.g., samples obtained from a bank of samples, e.g., IMPATH (BioClinical Partners, Inc, Franklin, Mass.). Generally, subjects with known medical history are chosen. Once the mass of the glycans on the target proteins in the samples has been determined and associated with the identity of those glycans, a correlation is made between the mass-identity of the glycans and the state or stage of the disease. As one example, changes in glycosylation may be correlated with disease state, including but not limited to the following: non-cancerous normal, non-cancerous hyperplastic (e.g., benign prostate hyperplasia (BPH)), non-cancerous inflammatory (e.g., prostatitis, proliferative inflammatory atrophy (PIA)), pre-cancerous (e.g., prostate intraepithelial neoplasia (PIN)), or cancerous (e.g., prostate cancer (PCa)). Changes in glycosylation may also be correlated with disease stage, for example using a system such as the TNM (tumor only (T), spread to a node (N), or metastatic (M)) or other grading system (including but not limited to the Gleason Grade/Gleason Score or other grading system. Taking prostate cancer as one example, which is not meant to be limiting, the following grading system may be useful: Stage I (A) cancer can't be felt on digital rectal exam (DRE), causes no symptoms, and has not spread outside the prostate; Stage II (B) cancer can be felt on DRE or increased PSA, but has not spread outside the prostate; Stage III (c) cancer has spread outside the prostate to nearby tissues; Stage IV (D) cancer has spread to lymph nodes or to other parts of the body. Any other system of staging disease, e.g., clinically or pathologically, that is known in the art can be used.

### Example 1

### Glycotyping PSA in LNCaP Cells

Isolation of PSA from LnCaP Cells:

LnCaP cells were plated in RPMI 1640 medium containing 10% FBS for 48-72 hours, and the cultures were washed with warm HBSS after which new medium was added. Culture supernatants were collected 24-48 hours later and frozen at ⁻20° C. PSA measurements were made on thawed supernatants using a commercially available mouse anti-human PSA monoclonal antibody (TandemE PSA Immunoenzymatic Assay; Hybritech, San Diego, Calif.). The results are generally expressed as ng/ml of PSA/10⁶ cells. The limit of sensitivity of this assay is approximately 0.2 ng/ml (Ballangrud et al., Clin Cancer Res 5:3171s-3176s (1999); Corey et al., Prostate 35:135-43 (1998); Gau et al., Cancer Res 57:3830-4 (1997); Hedlund et al., Prostate 41:154-65 (1999); Nagasaki et al., Clin Chem 45:486-96 (1999)).

Briefly, PSA from the media was purified by use of anti-PSA antibody linked gel. A polyclonal rabbit anti human PSA antibody (Donn et al., Prostate 14, 237-49 (1989)) (AXL 685, Accurate Chemical & Scientific Corporation) was linked to Protein G Sepharose using an Immunopure crosslinking kit (Pierce, Rockford, Ill.). Before crosslinking, protein G Sepharose was equilibrated with Immunopure binding buffer and then mixed with anti PSA IgG at a concentration of 3-4 mg IgG/ml of gel. The solution was mixed by gentle inversion at room temperature. After 30-60 minutes, the gel was washed with buffer and the antibody bound using a solution of DMP (Dimethyl pimelimidate) for 1-2 hours at room temperature; the remaining active sites was blocked using immunopure blocking buffer. Unbound IgG was eluted with glycine-HCl (pH 2.5), the gel was washed and then stored in PBS containing 0.02% sodium azide. For immunopurification, medium containing PSA was incubated with washed anti-PSA bound gel. After incubation at room temperature for 30 to 60 minutes, the unbound fraction was withdrawn and the gel was washed 3-4× with PBS. Bound PSA was then eluted in a batchwise procedure using an equal volume of 100 mM acetic acid. Resulting fractions (3 or 4) were collected and concentrated using a Speed Vac. Concentrated fractions were then resolved by SDS-PAGE to confirm purity and molecular size, as is shown in FIG. 4. In some cases, the fractions eluted were placed in tubes containing 50 ml of Tris-HCl (pH 8.5) and used for estimating concentrations of recovered PSA (Hybritech kit) (Qian et al., Clin. Chem. 43:352-9. (1997)).

Following isolation, the PSA is analyzed as described herein.

### Example 2

### Glycotyping PSA in Human Serum

Isolation of PSA from human serum:

A method of solid-phase affinity capture has been developed that is estimated to purify greater than 90% of the PSA present in serum samples (Hurst et al., Anal. Chem. 71:4727-33. (1999)). All reactions were carried out in sterile, low retention, 1.5 mL microcentrifuge tubes (VWR). Amino-polystyrene beads (3-3.4 mm, 5% w/v; Spherotech, Inc.) were treated with 0.5% glutaraldehyde in sodium carbonate buffer. After washing to remove excess reagent, a rabbit anti-human PSA antibody (Accurate Chemical & Scientific Corporation) in carbonate buffer was allowed to bind for several hours at room temperature. After washing the beads, 10 mg/mL sodium cyanoborohydride was allowed to react for 1 hour to covalently lock the antibody in place. The derivatized beads were mixed with human serum for 2 hrs. at room temperature with gentle rocking. After capture, the samples were washed 5× with PBS and eluted with 1:3:2 Formic Acid/Water/Acetonitrile.

Following isolation, the intact PSA was analyzed as described herein. FIG. 5 shows that PSA isolated from normal human serum and analyzed by the present methods is a relatively pure, single entity, with an empirically determined mass of 28,478.3 Da, which is in very close agreement with the theoretical molecular mass of the primary PSA polypeptide with a single fucosylated, biantennary sugar structure. These results illustrate that the present methods are applicable to PSA isolated from human serum. Similar methods can be used to isolate any target marker protein of choice, for instance, AFP or CEA.

### Example 3

### MALDI-MS Based Sequencing of N-linked Glycans from PSA

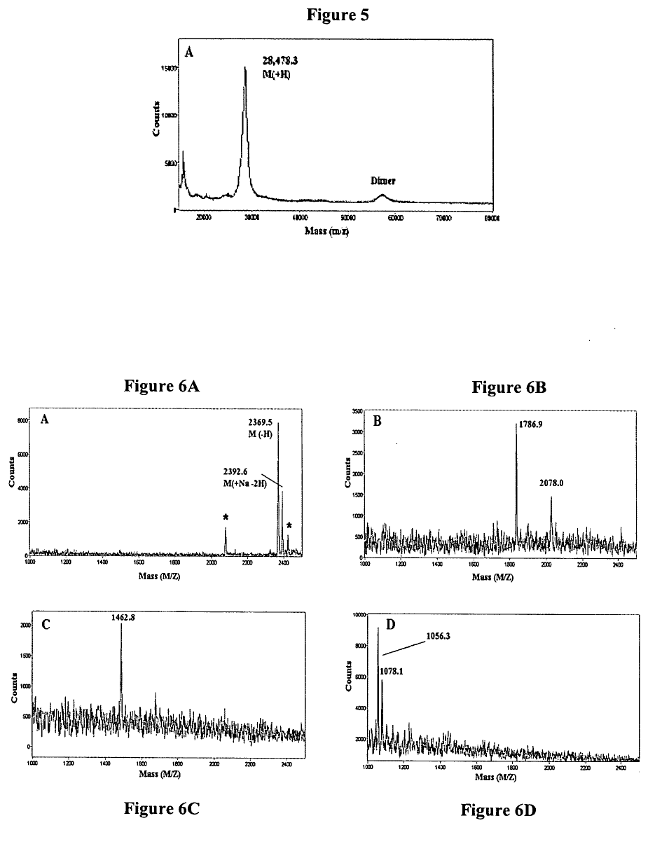

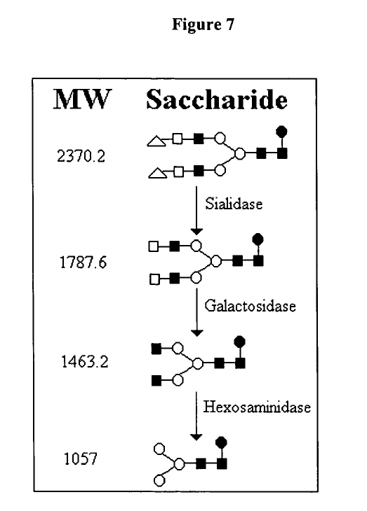

Normal PSA was obtained from Calbiochem or purified from serum samples of healthy male volunteers (obtained from a clinical organization called IMPATH) and the glycans were separated from the protein as described herein. Briefly, the glycan structure of PSA was isolated after PNGase F digestion and directly analyzed via MALDI-MS. As is shown in FIG. 6A, analysis of the intact glycan structure yielded a mass of 2369.5, which is consistent with a biantennary structure with a core fucose and two terminal sialic acids (theoretical mass of 2370.2; FIG. 7). Treatment with sialidase resulted in a decrease in mass of 582.6, consistent with the loss of two sialic acid residues (FIG. 6B and FIG. 7). The addition of galactosidase to the asialo sample resulted in a further mass decrease of 324.1, resulting from the cleavage of two galactose residues from the nascent chain (FIG. 6C and FIG. 7). Finally, treatment of the sample with N-acetylglucosaminidase resulted in a mass decrease consistent with the loss of two N-acetylglucosamine residues (FIG. 6D and FIG. 7).

These results demonstrate that the present methods are applicable to the determination of the composition of N-linked glycans from PSA. In addition, based on the enzyme specificity as well as the mass shift observed upon enzymatic treatment, the sequence of the unknown oligosaccharide can be determined, i.e., an unambiguous oligosaccharide structure can be assigned to unknown samples. These results confirms that the present methods are useful for providing the structural information required to assign mass identity relationships. In addition, this experiment was completed on submicrogram amounts of material, amounts available from in vivo samples, demonstrating the applicability of these methods to physiological samples. Similar methods can be used to determine the glycotype of any target protein, e.g., AFP and CEA.

### Example 4

### Comparison of PSA Glycotypes from Normal Individuals and Cancer Patients

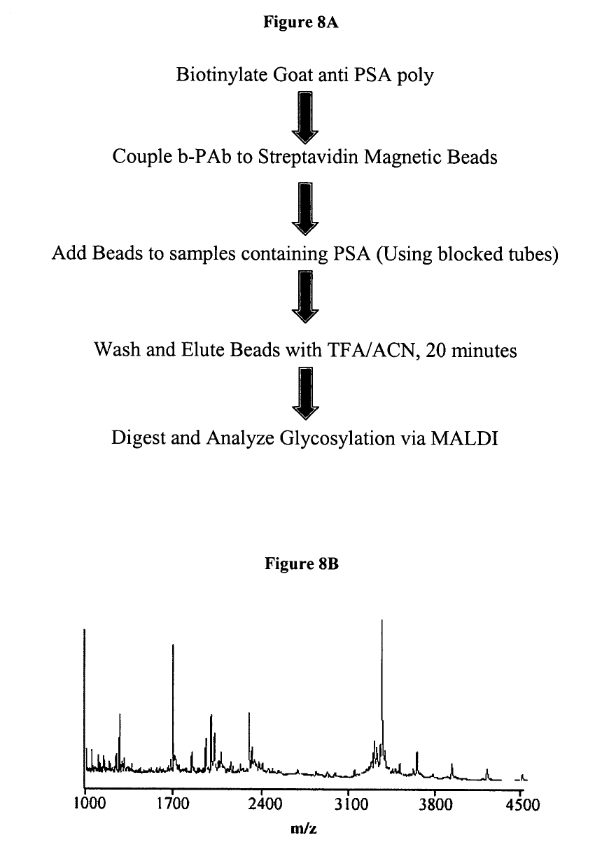

PSA from individuals suffering from prostate cancer was isolated from 1 mL serum samples as outlined in FIG. 8A. Briefly, PSA was captured on magnetic beads (Millipore Corp.) that were coated with a low affinity polyclonal antibody (Scripps Labs San Diego, Calif.). PSA was eluted with a 100% acetonitrile/0.1% TFA solution, and either analyzed as is or the glycan was analyzed separately after digestion. Typical yields after immunopurification were 60-80% as measured by an anti-PSA ELISA (Table 2). In some experiments, the PSA protein+glycosylation was analyzed directly via MALDI MS as outlined in Example 2. In these experiments, PSA from cancer patients consisted of multiple entities, many of which possessed a molecular mass greater than 28.5 kDa. Alternatively, the glycosylation of PSA was cleaved using either enzymatic (using PNGase, as described in Example 3) or chemical (using hydrazinolysis, substantially as described in Wolff et al. Prep Biochem Biotechnol. 29(1):1-21 (1999)) means.

The results of analysis after digestion with PNGase are shown in FIG. 8B. Direct analysis of the N-linked glycan of PSA in samples from individuals with cancer indicated that it is not the same as that found in normal PSA (i.e., PSA in samples from normal individuals that don't have cancer, compare FIGS. 6A and 8B), possessing species with a higher degree of branching as well as other modifications (i.e., see peak at about 3300 in FIG. 8B, which is not present in samples from normal individuals). Examination of both the mass of the intact PSA glycoprotein as well as the isolated glycan revealed that the glycoforms of PSA from cancer samples have several key differences, including (1) the PSA glycoforms in cancer possess a higher degree of branching as well as sialic acid; (2) the PSA glycoforms in cancer possess different fucosylated structures; and (3) chain length of antennary arms in PSA from cancer is distinct from that in normal individuals. Some samples from cancer patients also display improperly processed, lower molecular weight glycans as well (FIG. 8C). Of note is the fact that the two analyses, of intact and isolated glycans, gave separate but complimentary information.

TABLE 2
Typical yields of PSA from the serum of cancer patients.

| | PSA | PSA | % |
|---|---|---|---|
| Ab (mgs) | remain (ng) | elute (ng) | Recovery |
| 25 | 0 | 717 | 71.7 |
| 25 | 0 | 753 | 75.3 |
| 50 | 0 | 681 | 68.1 |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for evaluating the clinical status of a subject, the method comprising:
providing a sample from the subject, said sample comprising a pre-selected target glycoprotein; and
determining the glycoprofile of the target glycoprotein using a method that can detect a target glycoprotein in amounts less than 1000 ng/ml,
wherein the glycoprofile indicates that the subject has a predefined clinical status.

2. A method for evaluating a subject, the method comprising:
providing a sample comprising:
i. about 0.1 ng/ml to 1 μg/ml;
ii. about 5 pM to 50 nM;
iii. about 5 femtomoles/ml to 50 picomoles/ml;
iv. less than about 1 μg; or
v. less than about 50 pmols,
of a pre-selected target glycoprotein; and
determining the glycoprofile of the target glycoprotein,
wherein the glycoprofile indicates that the subject has a predefined clinical status.

3. A method for evaluating the clinical status of a subject, the method comprising:
providing a sample from the subject;
isolating a pre-selected target glycoprotein by immunopurification;
contacting the target glycoprotein with an enzyme; and
determining the glycoprofile of the target glycoprotein,
wherein the glycoprofile indicates that the subject has a predefined clinical status.

4. The method of claims 1-3, wherein the sample is concentrated before the glycoprofile is determined.

5. The method of claims 1-3, wherein the sample comprises urine, blood, serum, semen, saliva, feces, or tissue.

6. The method of claims 1-3, wherein the target glycoprotein is a marker for cancer.

7. The method of claims 1-3, wherein the target glycoprotein is selected from the group consisting of PSA, AFP, and CEA.

8. The method of claim 7, wherein the target glycoprotein is PSA.

9. The method of claim 1, wherein the glycoprofile of the target glycoprotein is determined using a method that can detect a target glycoprotein in amounts less than 500 ng/ml.

10. The method of claim 1, wherein the glycoprofile of the target glycoprotein is determined using a method that can detect a target glycoprotein in amounts less than 250 ng/ml.

11. The method of claim 1, wherein the glycoprofile of the target glycoprotein is determined using a method that can detect a target glycoprotein in amounts less than 100 ng/ml.

12. The method of claim 1, wherein the glycoprofile of the target glycoprotein is determined using a method that that can detect a target glycoprotein in amounts less than 10 ng/ml.

13. The method of claims 1-3, wherein the predefined clinical status is a stage of a disorder.

14. The method of claims 1-3, wherein the predefined clinical status is a stage of a cancer.

15. The method of claims 1-3, wherein the predefined clinical status is selected from the group consisting of cancer, a precancerous condition, a benign condition, and no condition.

16. The method of claims 1-3, wherein determining the glycoprofile comprises removing one or more pre-selected glycans from the target glycoprotein.

17. The method of claim 16, wherein the glycans are removed enzymatically.

18. The method of claim 16, wherein the glycans are removed using an enzyme selected from the group consisting of PNGase F, PNGase A, EndoH, EndoF, and O-glycanase.

19. The method of claim 16, wherein the glycans are removed using a protease.

20. The method of claim 16, wherein the glycans are removed using trypsin or LysC.

21. The method of claim 16, wherein the glycans are removed chemically.

22. The method of claim 16, wherein the glycans are removed using anhydrous hydrazine, reductive beta-elimination, or non-reductive beta-elimination.

23. The method of claims 1-3, wherein the determining comprises applying one or more experimental constraints to a glycan associated with the target glycoprotein.

24. The method of claim 23, wherein the experimental constraint is enzyme or chemical digestion of the glycan.

25. The method of claim 16, further comprising applying one or more experimental constraints to the glycan.

26. The method of claim 25, wherein the experimental constraint is enzyme or chemical digestion of the glycan.

27. The method of claims 1-3, wherein determining the glycoprofile comprises determining one or more of: the presence, concentration, percentage, composition, or sequence of one or more glycans associated with the target glycoprotein.

28. The method of claims 1-3, further comprising repeating one or more of the steps.

29. The method of claims 1-3, wherein the sample comprises less than 50 pmol of the selected target molecule.

30. The method of claims 1-3, wherein the sample comprises less than 10 pmol of the selected target molecule.

31. The method of claims 1-3, wherein the sample comprises less than 1.0 pmol of the selected target molecule.

32. The method of claims 1-3, wherein the sample comprises less than 0.5 pmol of the selected target molecule.

33. The method of claims 1-3, wherein the sample comprises less than 0.1 pmol of the selected target molecule.

34. The method of claims 1-3, wherein the sample comprises less than 0.05 pmol of the selected target molecule.

35. The method of claims 1-3, wherein the sample comprises less than 0.01 pmol of the selected target molecule.

36. The method of claims 1-3, wherein the sample comprises less than 0.005 pmol of the selected target molecule.

37. The method of claims 1-3, wherein the determining is by a method selected from CE, CE/LIF, NMR, MALDI mass spectrometry, ESI mass spectrometry, and HPLC with fluorescence detection.

38. The method of claim 37, wherein the determining is by CE/LIF.

39. The method of claim 37, wherein the determining is by MALDI-MS.

40. The method of claims 1-3, wherein the subject is suspected of having a cellular proliferative and/or differentiative disorder.

41. The method of claim 40, wherein the disorder is cancer.

42. The method of claim 41, wherein the cancer is selected from the group consisting of carcinoma, sarcoma, metastatic disorders and hematopoietic neoplastic disorders.

43. The method of claim 41, wherein the hematopoietic neoplastic disorder is a leukemia.

44. The method of claims 1-3, wherein the glycoprofile indicates that the subject has cancer.

45. The method of claims 1-3, wherein the glycoprofile indicates that the subject has a pre-disorder condition.

46. The method of claim 45, wherein the pre-disorder condition is a precancerous condition.

47. The method of claims 1-3, wherein the glycoprofile indicates that the subject has a benign condition.

48. The method of claim 47, wherein the benign condition is a benign tumor or a benign hyperplasia.

49. The method of claim 48, wherein the benign hyperplasia is benign prostatic hyperplasia (BPH).

50. The method of claim 26, wherein the presence, concentration, percentage, composition, or sequence of one or more glycans indicates that the subject has cancer.

51. The method of claim 26, wherein the presence, concentration, percentage, composition, or sequence of one or more glycans indicates that the subject has a pre-cancerous condition.

52. The method of claim 50, wherein the cancer is breast carcinoma, lung carcinoma, colon carcinoma, prostate cancer or hepatocellular carcinoma.

53. The method of claim 44, wherein the cancer is breast carcinoma, lung carcinoma, colon carcinoma, prostate cancer or hepatocellular carcinoma.

54. The method of claim 50, wherein the presence, concentration, percentage, composition or sequence of one or more glycans further indicates the stage of the cancer.

55. The method of claim 50, wherein the presence, concentration, percentage, composition or sequence of one or more glycans further indicates the growth rate of the cancer.

56. The method of claim 50, wherein the presence, concentration, percentage, composition or sequence of one or more glycans further indicates prognosis.

57. The method of claims 1-3, wherein the subject does not have cancer.

58. The method of claim 57, wherein the subject has one or more benign hyperplasias.

59. The method of claim 58, wherein the benign hyperplasia is benign prostatic hyperplasia.

60. The method of claims 1-3, wherein the subject has a precancerous condition.

61. The method of claims 1-3, wherein the subject has a PSA level of 0-4 ng/mL, 4-10 ng/mL, 10-20 ng/ml, or >20 ng/ml.

62. The method of claims 1-3, wherein the subject is being screened for a disorder associated with changes in the glycoprofile of a target glycoprotein.

63. The method of claim 62, wherein the disorder is a cellular proliferative or differentiative disorder.

64. The method of claim 63, wherein the disorder is cancer.

65. The method of claim 62, wherein the subject has previously tested negative for the disorder by another, non-sugar based diagnostic method.

66. The method of claim 65, wherein the non-sugar based diagnostic method is one or more of physical examination, immunodiagnostic test, detection of protein levels, imaging, or biopsy.

67. The method of claim 66, wherein the detection of protein levels is in blood or urine.

68. The method of claim 66, wherein the imaging method is selected from the group consisting of x-ray, MRI, CAT, and ultrasound.

69. The method of claims 1-3, wherein a second, non-glycoprofile diagnostic test is also performed.

70. The method of claim 69, wherein the non-glycoprofile diagnostic test is performed at one or more of: before with, concurrently with, or after the glycoprofile determination.

71. The method of claims 1-3, further comprising
providing a reference; and
comparing the glycoprofile of the target molecule to the reference.

72. The method of claim 71, wherein comparing the glycoprofile comprises one or more of:
comparing the presence, concentration, percentage, composition or sequence of one or more selected glycans of the target molecule to the reference.

73. The method of claim 71, wherein the comparing allows staging or prognosis.

74. A method for monitoring a subject, the method comprising:
(a) providing a sample from the subject comprising a target glycoprotein;
(b) purifying the target glycoprotein;
(c) contacting the target glycoprotein with an enzyme;
(d) determining the glycoprofile of the target glycoprotein; and
(e) repeating steps a-d one or more times.

75. The method of claim 74, wherein the repeating is done after administration of a treatment to the subject.

76. The method of claim 74, wherein the enzyme is immobilized.

77. The method of claim 3, wherein the enzyme is immobilized.

78. A method of determining the metastatic potential of a tumor, the method comprising:
providing a sample from the subject;
isolating a target protein by immunopurification;
contacting the target protein with immobilized enzyme; and
determining the glycoprofile of the target protein,
wherein the glycoprofile indicates the metastatic potential of the tumor

79. A database comprising a plurality of records, wherein each record includes one or more of the following:
(a) data on the glycoprofile of a target glycoprotein associated with a disorder isolated from a sample from a subject;
(b) data on the status of the subject;
(c) data on any treatment administered to the subject;
(d) data on the subject's response to treatment;
(e) personal data on the subject; and
(f) environmental data.

80. The method of claim 79, wherein the data on the status of the subject comprises information regarding whether the subject has cancer, a pre-cancerous condition, a benign condition, or no condition.

81. The method of claim 79, wherein the data on the status of the subject comprises information regarding the clinical status of the subject's disorder.

82. The method of claim 81, wherein the clinical status of the subject's disorder comprises in remission, recurring, recovered, cured, improved, metastasized, chronic, or terminal.

83. The method of claim 79, wherein the data on the subjects' response to the treatment includes information regarding one or more of the efficiency of the treatment side effects.

84. The method of claim 79, wherein the data on the treatment includes information regarding one or more of: any drug administered; dosages; dosing schedules; and compliance.

85. The method of claim 79, wherein the personal data on subject includes information regarding one or more of: age; gender; education; medical history; and family medical history.

86. The method of claim 79, wherein the environmental data includes information regarding one or more of: the presence of a substance in the environment; residence in a preselected geographic area; and performing a preselected occupation.

87. A method of evaluating a subject, the method comprising
providing a sample from a subject
immunopurifying a target protein from the sample; and
determining the glycoprofile of the target protein in the sample,
wherein the glycoprofile of the target protein in the sample indicates that the subject has cancer, a precancerous condition, or a benign condition.

88. A method of evaluating a subject, the method comprising:
providing a sample from the subject
immunopurifying PSA from the sample; and
determining the glycoprofile of the PSA in the sample,
wherein the glycoprofile of the PSA in the sample indicates that the subject has or does not have cancer or benign prostate hyperplasia.

89. The method of claim 88, wherein the subject has serum PSA levels of 0-4 ng/mL, 4-10 ng/mL; 10-20 ng/ml; or >20 ng/ml.

90. The method of claim 89, wherein the subject has serum PSA levels of 0-4 ng/mL.

91. The method of claim 89, wherein the subject has serum PSA levels of 4-10 ng/mL.

92. The method of claim 89, wherein the glycoprofile includes the presence of a high molecular weight glycan that is not present in a sample from a subject who does not have cancer, and indicates that the subject has cancer.

93. The method of claim 92, wherein the high molecular weight glycan has a molecular weight of about 3300.

94. A method of evaluating a subject, the method comprising:
providing a sample from said subject;
immunopurifying AFP from the sample; and
determining the glycoprofile of the AFP,
wherein the glycoprofile of the AFP indicates that the subject has or does not have cirrhosis or HCC.

95. The method of claim 95, wherein the subject has serum AFP levels of 0-20 ng/mL; 20-1000 ng/mL; or >1000 ng/ml.

96. A method of evaluating a subject, the method comprising:
providing a sample from said subject;
immunopurifying CEA from the sample; and
determining the glycoprofile of the CEA,
wherein the glycoprofile of the CEA indicates that the subject has or does not have a cancer of the colon, stomach, lung, pancreas, liver, breast, or esophagus.

97. The method of claim 96, wherein the subject has serum or plasma CEA levels of 0-5 ng/mL; 5-10 ng/mL; >10 ng/ml.

98. The method of claims 1-3, wherein determining the glycoprofile comprises detecting one or more of: alterations in sialylation, modification of sialic acids, sulfation, branching, presence or absence of a bisecting N-acetylglucosamine, and changes in the number of glycosylation sites.

99. The method of claims 1-3, wherein determining the glycoprofile comprises detecting alterations in β1-6 branching structures, of one or more of N-linked and O-linked oligosaccharides

100. The method of claims 1-3, wherein determining the glycoprofile comprises detecting one or more of alterations in Lewis antigens, sialylation, and fucosylation.

101. A method of evaluating the status of a subject, the method comprising:
providing a sample from the subject;
immunopurifying a pre-selected target protein from the sample using antibodies bound to magnetic beads;
contacting the purified target protein with immobilized enzyme; and
determining the glycoprofile of the target protein,
wherein the glycoprofile indicates the status of the subject.

102. A method for identifying candidate reagents capable of detecting glycoprofile differences between a first glycoprotein having a first glycoprofile and a second glycoprotein having a second glycoprofile, wherein one or both glycoproteins is present in less than 50 pmols, the method comprising:
contacting the first glycoprotein with one or more candidate reagents;
optionally contacting the second glycoprotein with the one or more candidate reagents; and
evaluating the ability of the candidate reagents to detect glycoprofile differences between the first and second glycoproteins.

103. The method of claim 102, wherein the one or more candidate reagents are selected from the group consisting of lectins, antibodies, and polysaccharide-binding peptides.

104. The method of claim 103, wherein the polysaccharide-binding peptides are isolated through phage display.

105. The method of claim 102, further comprising determining the glycoprofile of the first glycoprotein.

106. The method of claim 102, further comprising determining the glycoprofile of the second glycoprotein.

107. The method of claim 102, wherein the first and second glycoproteins are obtained from subjects having different clinical statuses.

108. The method of claim 107, wherein the different clinical statuses include normal, having a benign hyperplastic disorder, having a precancerous disorder, having cancer, having a metastatic cancer, in remission, recovered from cancer, recovered from a precancerous disorder, recovered from a metastatic cancer, and deceased.

109. The method of claim 102, wherein the first and second glycoproteins have the same protein core.
